(19) United States
(12) Patent Application Publication
(10) Pub. No.: US 2011/0060322 A1
(43) Pub. Date: Mar. 10, 2011

(54) APPARATUS AND METHOD FOR SURFACE COOLING

(75) Inventor: Dieter Manstein, Miami, FL (US)

(73) Assignee: The General Hospital Corporation, Boston, MA (US)

(21) Appl. No.: 12/934,926

(22) PCT Filed: Mar. 26, 2009
(86) PCT No.: PCT/US09/38422

§ 371 Date: Nov. 8, 2010

Related U.S. Application Data
(60) Provisional application No. 61/040,053, filed on Mar. 27, 2008.

(51) Int. Cl. A61B 18/18

(52) U.S. Cl. 606/9

(57) ABSTRACT

Exemplary embodiments of the present disclosure provide method and apparatus for cooling a tissue surface that includes a housing configured to enclose a volume above a target region of the tissue, at least one outlet arrangement (e.g., duct) through which gas can be withdrawn from the enclosed volume, and at least one inlet arrangement (e.g., duct) that allows or facilitates further gas to enter the enclosed volume. A flow of gas can thus be provided over the target area that can cool it by convection. A liquid can be provided on the target area to provide additional evaporative cooling. Electromagnetic energy can be directed onto the target area through a portion of the housing, and debris generated by interaction of the energy with the tissue can be safely contained within the enclosed volume and removed therefrom through the outlet duct.

### CROSS REFERENCE TO RELATED APPLICATION

The present application claims priority from U.S. Provisional Patent Application Ser. No. 61/040,053 filed Mar. 27, 2008, the disclosure of which is incorporated herein by reference in its entirety.

### FIELD OF THE INVENTION

The present disclosure relates to cooling of a surface region, such as a biological tissue surface, and in particular to method and apparatus which can facilitate such cooling.

### BACKGROUND INFORMATION

There is an increasing demand for repair of skin defects that can be induced by aging, sun exposure, dermatological diseases, heredity, traumatic effects, and the like. For example, some treatment techniques use electromagnetic radiation to generate thermal and/or damage to the skin, which can result in a wound healing response that leads to a biological repair of the damaged skin or other desirable effects. Electromagnetic radiation provided, e.g., by a laser, an intense pulsed light source (“IPL”), a flashlamp, or the like can also be used for hair removal.

Application of an electromagnetic radiation to treat skin tissue may often be accompanied by undesirable side effects, including a sensation of pain in the patient being treated. A reduction of pain sensation can be achieved, e.g., by cooling the region of skin tissue being treated before and/or during the treatment. Cooling of skin tissue can also increase the ratio of ablation depth to a thermal affected zone diameter during a laser ablative procedure. For example, a cooled tissue can facilitate targeting of deeper tissue with an ablative laser while reducing thermal damage in adjacent regions along the surface thereof.

Techniques for cooling skin tissue can be based on various physical mechanisms. For example, conductive cooling can be achieved by contacting a cold object with the surface of the skin tissue. However, conductive cooling can obstruct electromagnetic radiation being directed to the skin tissue. To avoid this problem, the cold object can be formed of a material which does not significantly absorb or reflect the particular electromagnetic radiation being provided. It is noted that this requirement can limit the choice of materials which can be used to cool the skin surface, and such materials may not have sufficient thermal capacity to provide effective cooling. Accordingly, conductive cooling may be performed on a target region of skin tissue prior to treatment of the tissue with electromagnetic radiation, and may not be suitable for cooling skin tissue during such treatment.

Convective cooling can also be used cool a target region of skin tissue by directing a fluid (e.g., a gas) over the target region. Motion of the fluid relative to the tissue surface increases the effective heat transfer coefficient between the tissue and the fluid to enhance the rate of skin tissue cooling. The fluid may optionally be cooled to provide increased cooling. Such flowing fluid may facilitate the electromagnetic radiation to pass therethrough relatively unimpeded, such that convective cooling can be used effectively during treatment of the skin tissue. Systems configured to provide convective cooling may require large cooling arrangements to cool the continuously flowing gas, and thus may be bulky and/or inefficient to operate.

Convective cooling may be accompanied by evaporative cooling, where the evaporation of a liquid on the surface being cooled is enhanced by the moving fluid (e.g., gas). Release of the enthalpy of vaporization when the surface liquid evaporates can provide further cooling of the surface. Such liquid may be naturally present, e.g. sweat or perspiration, and/or it may be applied to the surface being cooled. Other liquids, such as alcohols, which tend to evaporate quickly, may be applied to the surface being cooled to increase the rate of cooling. The use of evaporative cooling can be limited by the ability to provide and/or maintain an evaporating liquid on the surface being cooled.

Another conventional technique which may be used to cool skin tissue uses a cryospray, which is a cold vapor that is directed to the surface to be cooled. Conventional cryosprays include a cryogenic substance in a liquid form that is maintained under pressure in a container. The cryogenic substance may be present in a gaseous state under normal (e.g., atmospheric) pressure. When the cryogenic substance is controllably released from the container, such as through a valve or nozzle, it converts to a vapor and expands under the lower pressure outside the container, and may cool significantly by operation of the Joule-Thompson effect. Such cryosprays can provide a cold stream of vapor that can be directed to the surface to be cooled. Because the expanded vapor can be very cold, cryospray techniques may be used to generate a series of short bursts of the cold vapor to cool an object. However, it is difficult to maintain a moderate and continuous degree of cooling using cryosprays. For example, portions of skin tissue may freeze when exposed to a cold cryospray for too long of a time.

The electromagnetic radiation can be directed onto skin tissue using, e.g., certain types of lasers, to cause ablation of tissue. Ablation generally removes a portion of the tissue exposed to the electromagnetic radiation by vaporization and/or evaporation of tissue components. The laser ablation process can form a plume containing debris from the removed tissue. An object placed over a target area of tissue being ablated, e.g., a conductive cooling mass, may prevent some or all of the plume from being released from the tissue that is treated. This can lead to a dangerous buildup of debris and heat in the target region.

The plume formed during tissue ablation can also produce undesirable effects. For example, the plume may interfere with a beam of the electromagnetic radiation provided by the laser, causing partial reflection, absorption, and/or diffusion of the applied beam. The debris itself may also present a health hazard. For example, ablated debris may contain infectious material (e.g., a virus or bacteria), and allowing such debris to enter the environment may be harmful.

Accordingly, there may be a need to address and/or overcome at least some of the deficiencies or issues described herein above.

### SUMMARY OF EXEMPLARY EMBODIMENTS

Exemplary embodiments of the present disclosure provide an apparatus for cooling a surface, such as, e.g., a target region of skin tissue, by facilitating a flow of gas over the surface. This cooling can be performed, e.g., during treatment of the tissue by an exposure to an electromagnetic radiation, such as optical energy from a laser or an IPL. Such cooling can reduce the perception of pain during the treatment of the skin tissue. The exemplary embodiment of the apparatus can further provide containment and removal of debris which can be generated from the tissue during treatment, such as a plume formed by exposing tissue to an ablative laser.

According to one exemplary embodiments of the present disclosure, a cooling apparatus can be provided that can include a housing with an opening on a lower portion thereof that is configured to enclose a volume over the target region of tissue. In certain exemplary embodiments, the housing can includes one or more windows that facilitates an electromagnetic radiation to pass therethrough with substantially no interaction with the window, and/or that allow or facilitate for a visual observation of the target region from outside the housing. In other exemplary embodiments, the housing can be formed partially or entirely of a material which allows visual observation of the target region and/or transmission of the electromagnetic radiation therethrough. The exemplary apparatus can include one or more inlet arrangements (e.g., ducts) connected to the housing that are configured to facilitate a gas to enter the volume enclosed by the housing, and one or more outlet arrangements (e.g., ducts) that facilitate the gas to be removed from the volume.

In still other exemplary embodiments of the present disclosure, the housing can include a hole or an opening on an upper surface thereof that facilitates or allows energy, e.g., a laser radiation, to enter therethrough. For example, an opening may be provided in a portion of the housing that is configured to allow or facilitate a portion of an optical guide or outlet aperture of a laser to be attached thereto. Such exemplary configuration can allow or facilitate the energy to be directed onto the surface without passing through the housing material. In certain exemplary embodiments, a source of directed energy, e.g., a laser aperture or optical waveguide, can be directly coupled to the housing or configured to pass through a portion of the housing, e.g., forming a substantially airtight seal with the housing. The source of directed energy can be movably attached to the housing, such that the energy can be directed to various locations on the surface without moving the housing.

A portion of the exemplary housing can also be formed of a material that allows or facilitates an observation of the target region and of an energy-tissue interaction during treatment. The exemplary housing can also be formed using a material can further reduce or eliminate emission of harmful radiation from beneath the housing (e.g., back-reflected radiation from a CO₂ laser).

In certain further exemplary embodiments, the apparatus can include a source of low pressure or vacuum configured to pull a gas in from the one or more inlets and out from the volume enclosed by the housing through the outlet duct, e.g., creating a flow of the gas over the surface of the target region. The low pressure source can include a pump arrangement, an evacuated vessel or container, or the like. Such flow can cool the tissue surface by convective and/or evaporative cooling. A filter can also be provided in the outlet duct to remove debris contained in the gas being removed from the volume, such as material in a plume generated by an ablative laser interacting with tissue in the target region.

In still further exemplary embodiments, a cooling arrangement can be provided to cool gas entering the volume enclosed by the housing through the inlet duct. A filter may also be provided in the inlet duct to remove contaminants or particulates in the gas before it enters the volume and flows over the target region. In certain exemplary embodiments of the present disclosure, the gas removed from the enclosed volume by the low pressure source can be directed to the cooling arrangement to increase the cooling efficiency thereof.

In still further exemplary embodiments, a valve can be provided in the inlet duct to control the flow of the gas through the inlet duct and into the volume enclosed by the housing, and to optionally provide a pulsed flow of the gas over the target region. Such pulsed flow can provide enhanced cooling and/or a reduction of pain sensation. A valve arrangement can also be provided at the outlet duct to control flow of gases exiting the enclosure.

The exemplary cooling apparatus can further include a spray nozzle coupled to the housing, and configured to direct a spray or stream of liquid onto the target region being cooled. Evaporation of such liquid by the gas flowing over the target region can provide an enhanced cooling by evaporation. The liquid can include water, an alcohol, an analgesic such as lidocaine, a bactericide or other biologically active substance, or any combination thereof. The spray nozzle, valve arrangements, and/or low pressure source can be controlled to provide alternating pulses of sprayed liquid and flowing gas to increase the effectiveness of the evaporative cooling. In certain exemplary embodiments, the gas flow within the enclosed volume and the liquid spray can each be continuous, e.g., while energy is being applied to the tissue.

In another exemplary embodiment of the present disclosure, a method can be provided using which a surface which includes providing a housing to enclose a volume over at least a portion of the surface can be cooled, and a gas out of the enclosed volume between the housing and the surface can be drawn out or removed through one or more outlet ducts to generate a flow of the gas over the surface. One or more inlet ducts can be provided in the housing to allow or facilitate further gas to enter the space enclosed by the housing and allow or facilitate the flow to be maintained for a desired period of time.

In further exemplary embodiments of the present disclosure, the flow can be pulsated to create a vibration in the surface being cooled, which can further reduce a sensation of pain if the surface is associated with a biological tissue such as skin. The pulsated flow can be provided by controlling a pump used to draw gas from the enclosed volume, and/or by controlling one or more valves provided in the inlet and/or outlet ducts.

In still further exemplary embodiments, at least a portion of the gas can be cooled before entering the enclosed volume to provide additional cooling of the surface.

In yet further exemplary embodiments, the surface being cooled can be sprayed with a liquid, and evaporation of the liquid from the surface can provide additional cooling. Such evaporation may be enhanced by the flow of gas over the surface. The sprayed liquid can include water, alcohol, any other liquid which can be evaporated using the gas flow, or a mixture thereof.

These and other objects, features and advantages of the present disclosure will become apparent upon reading the following detailed description of embodiments of the invention, when taken in conjunction with the appended claims.

### BRIEF DESCRIPTION OF THE DRAWINGS

Further objects, features and advantages of the present disclosure will become apparent from the following detailed description taken in conjunction with the accompanying figures showing illustrative embodiments, results and/or features of the exemplary embodiments of the present disclosure, in which:

FIG. 1 is a schematic diagram of a cooling apparatus in accordance with exemplary embodiments of the present disclosure;

FIG. 2 is a schematic diagram of a cooling apparatus in accordance with further exemplary embodiments of the present disclosure;

FIG. 3 is a plan view of several exemplary shapes that can be used for an apparatus housing in accordance with exemplary embodiments of the present disclosure; and

FIG. 4 is a schematic diagram of a cooling apparatus in accordance with still further exemplary embodiments of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures. It is intended that changes and modifications can be made to the described embodiments without departing from the true scope and spirit of the present disclosure as defined by the appended claims.

### DETAILED DESCRIPTION OF EXEMPLARY EMBODIMENTS

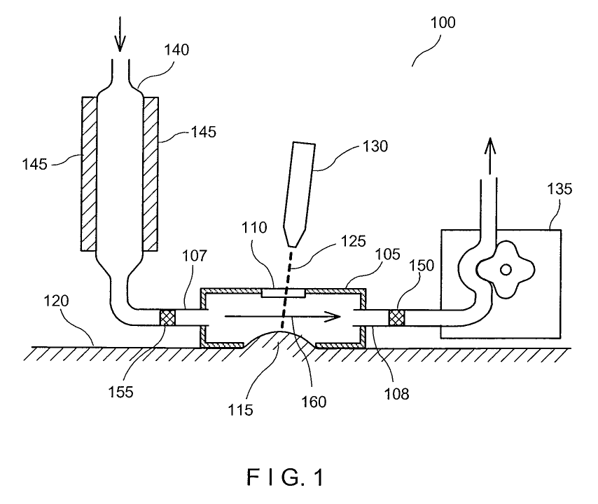

An exemplary embodiment of an apparatus 100 which may be used to cool a surface such as skin tissue is shown in FIG. 1. The exemplary apparatus 100 can include a housing 105 which is provided with at least one inlet duct 107 and at least one outlet duct 108. The housing 105 may include a window 110, which allows or facilitates an observation of a target area 115 of skin tissue 120 to be treated. The window 110 can also allow or facilitate an electromagnetic radiation 125 provided from an energy source 130 to pass therethrough and interact with the target area 115. A low-pressure source 135 may be provided in communication with the outlet duct 108. An optional cooling arrangement that can include, e.g., an enclosure 140 and a cooling device 145, can be connected to the inlet duct 107. An optional outlet filter 150 may be provided in the outlet duct 108, and an optional inlet filter 155 may be provided in the inlet duct 107.

The housing 105 of the exemplary apparatus 100 can be configured to be placed over the target region 115 of skin tissue 120 (or other tissue) to be treated, thereby enclosing a volume above the target region 115. A lower portion of the housing 105 can be configured to contact the surface of the skin tissue 120 surrounding the target region 115, such that a seal may be formed between the housing 105 and the tissue surface 120. A resilient material or gel can be provided on the lower portion of the housing 105 to improve contact between the housing 105 and the surface of the skin tissue 120 surrounding the target region 115. Such resilient material or gel, and/or one or more rollers or other low-friction arrangements can also be provided on the lower portion of the housing to facilitate translation of the housing 105 over the surface of the tissue 120. In further exemplary embodiments, an ink or other visible substance can be provided along at least a portion of the lower portion of the housing 105. Such ink can assist in placement of the housing 105 relative to the target area 115, and can also indicate which areas of the tissue 120 have been treated if the housing 105 is moved over the surface of the tissue 120.

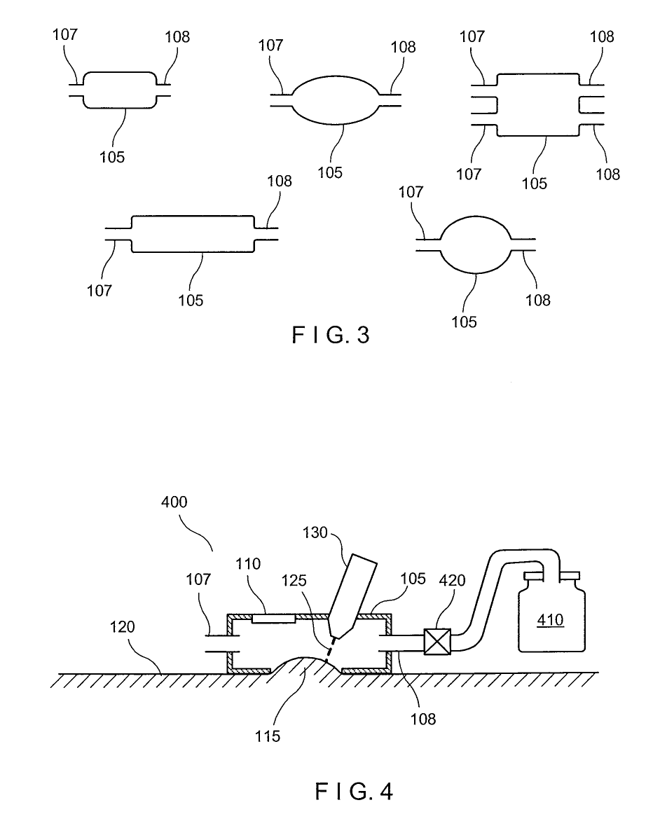

The housing 105 can be provided in any one of a variety of shapes. For example, when viewed in plan, the housing 105 can have a shape that is approximately square, rectangular, oval or ovoid, etc. The housing 105 can include sidewalls which extend from an upper surface of the housing 105 downward to the surface of the skin tissue 120. Alternatively, the housing 105 can have a contour of a shallow dome that may be round or oval as viewed in plan, or any other configuration that may be selected to cover the target region 115. The housing 105 can be sized and shaped such that it is large enough to surround an entire area of the tissue 120 to be treated with energy or electro-magnetic radiation. Alternatively, the housing 105 can be relatively small in size and configured to be moved over the surface of the tissue 120 to treat a plurality of areas of the tissue 120. Top or plan views of several exemplary housing shapes are shown in FIG. 3.

The housing 105 can have a low profile such that a dimension of the housing 105 along the surface is greater than a dimension thereof, e.g., perpendicular to the surface. This exemplary low profile can facilitate a maintenance of a flow of gas (indicated by arrow 160) that is substantially parallel to the surface of the target area 115. Such exemplary low profile can also facilitate an increase of a velocity of gas over the surface of the tissue 120 for a particular volumetric flow rate of the gas through the inlet duct 107 and/or outlet duct 108. A higher gas velocity can provide higher rates of cooling and/or evaporation from the tissue surface 120.

The window 110 can be provided in the upper surface of the housing 105 to allow or facilitate the electromagnetic radiation 125 from the energy source 130 to pass therethrough and interact with at least a portion of the skin tissue 120 located in the target region 115. The window 110 can be formed of or include a material which does not significantly interact or interfere with the electromagnetic radiation 125, and allows or facilitates such radiation 125 to pass therethrough with substantially no or little absorption or reflection. The window 110 can alternatively or additionally be configured to allow or facilitate visual observation of the target region 115 from above the housing 115. A plurality of such windows 110 may also be provided in the housing 115. In certain exemplary embodiments, the entire housing 105 or a substantial portion of the upper surface thereof, may be formed of a material which allows or facilitates a visual observation of the target region 115 and/or substantially unimpeded passage of the electromagnetic radiation 125 therethrough.

The inlet duct 107 and the outlet duct 108 are preferably located apart from each other, e.g., at opposed portions of the housing 105. Such spaced-apart placement of the inlet and outlet ducts 107, 108 can facilitate a flow of gas between the inlet duct 107 and the outlet duct 108 that passes over the target region 115. In certain exemplary embodiments, the housing 105 can be provided with a plurality of inlet ducts 107 and/or a plurality of outlet ducts 108.

The low-pressure source 135, such as, e.g., a vacuum source, can be connected to the outlet duct 108. The low-pressure source 135 can include, for example, a pump arrangement, such as a positive displacement pump or a vacuum pump, or any other device which can provide a source of low pressure capable of withdrawing gas from the volume enclosed by the housing 105. The flow or vacuum capacity of the low-pressure source 135 can be selected based on the size and configuration of the housing 135, the size and configuration of the inlet duct 107 and the outlet duct 108, the desired flow velocity over the target region 115 of the tissue 120, etc.

The low-pressure source 135 may be configured to remove gases and any entrained substances (e.g., debris from a plume) from the volume enclosed by the housing 105 through the outlet duct 108. For example, as gases are removed from this enclosed volume by the low-pressure source 135, additional gases can be pulled into the enclosed volume through inlet duct 107. In this manner, a flow of gas (indicated by the arrow 160 in FIG. 1) can be generated or induced over the target region 115. This flow of gas 160 can provide convective and/or evaporative cooling to the target region 115. Such cooling can be maintained while the target region 115 is exposed to electromagnetic radiation 125 from energy source 130.

Exemplary embodiments of the present disclosure described herein can provide certain benefits as compared to conventional cooling systems that blow or otherwise direct a flow of air or other gas over the tissue being treated. For example, using the low-pressure source 135 to pull gas from the volume enclosed by the housing 105 can help maintain contact between the housing 105 and the surface of the skin tissue 120. Such exemplary arrangement and/or configuration also can contain debris and/or other by-products formed when the tissue 120 is exposed to the electromagnetic energy 125, and allow them to be removed through the outlet duct 108 rather than being released into the surrounding environment.

Exemplary embodiments of the present disclosure may also allow or facilitate a more precise control of the flow velocity and geometry over the target region 115 through a suitable choice of the shape of the housing 105, size and placement of the inlet and outlet ducts 107, 108, control of the low-pressure source 135, etc.

The opening of the inlet duct 107 can be relatively small in size, such that gas flowing through the inlet duct 107 expands when it enters the volume enclosed by the housing 105. Such expansion can cool the gas as it enters the enclosed volume, which may further enhance the cooling of the target region 115 as the cooled gas flows over the target region 115.

The low-pressure source 135 can be a pumping arrangement that includes controller circuitry for turning the low-pressure source 135 on and off. The controller circuitry can also control the rate at which gases are extracted from the enclosed volume within the housing 115 (e.g., by varying the speed of the pump). The low-pressure source 135 may also generate a slight vacuum in the volume between the housing 105 and the target region 115. This lower pressure can pull the surface of the target region 115 upward into the volume enclosed by the housing 105, which can stretch the tissue 120 slightly. Such stretching can be beneficial when exposing the target region 115 to the electromagnetic energy 125, for example, by increasing the rigidity of the tissue and/or by promoting closure of any holes or incisions formed in the stretched tissue after the tissue 120 is allowed to relax.

The energy source 130 can include an intense pulsed light source, a laser, or any other energy source which can direct energy to the target region 115 to produce an interaction with a portion of the skin tissue 120. The energy source 130 can be an ablative laser, such as a CO₂ laser or an Er:YAG laser, which can ablate a portion of the tissue in the target region 115 and create a plume of debris. This debris and any other substances present in the volume enclosed by the housing 105 can be withdrawn through the outlet duct 108 by the low-pressure source 135 as described herein.

In further exemplary embodiments, the outlet filter 150 can be provided in communication with the outlet duct 108 to remove debris from the gases flowing into the outlet duct 108. Any conventional filter configuration may be used that is suitable for trapping and/or removing the debris from the flowing gas. For example, the outlet filter 150 may include a cartridge containing a fibrous or microporous medium, such that the cartridge can be periodically replaced as it becomes saturated with debris. The outlet filter 150 can be provided in the outlet duct 108 between the housing 105 and the low-pressure source 135 as shown in FIG. 1, or alternatively it may be provided at an outlet of the low-pressure source 135. A plurality of outlet filters 150 may also be provided.

The inlet filter 160 can be provided in the inlet duct 107 to remove particles or contaminants from gas entering the volume beneath the housing 105 and flowing over the target region 115. The inlet filter 155 can be similar in structure to the outlet filter 150, or it may have a different configuration. A plurality of inlet filters 155 can also be provided.

To enhance the convective cooling of the target region 115, the cooling arrangement can be connected to the inlet duct 107. For example, the cooling arrangement can include the enclosure 140 and the cooling device 145 that is configured to lower the temperature of the gas contained within and/or flowing through the cooling enclosure 140. The cooling device 145 can include, for example, one or more Peltier elements, one or more conduits containing a flowing coolant that are provided in contact with the cooling enclosure 140, a cooled bath surrounding a portion of the cooling enclosure 140, a phase-change medium, and the like. For example, the phase-change medium can be ice, dry ice, or the like. The cooling device 145 can include, for example, a cold object formed of a material having a large thermal mass.

The cooling enclosure 140 may have at least one small dimension (e.g., it can have the form of a narrow tube or flat channel) to improve contact between the cooling device 145 and the gas within the cooling enclosure 140, and thereby more effectively lower the temperature of the gas. The cooling device 145 can further include control circuitry and a temperature sensor provided, e.g., in the inlet duct 107 or adjacent to the enclosure 105 to facilitate a more precise control of the temperature of the gas flowing through the inlet duct 107 and over the target region 115.

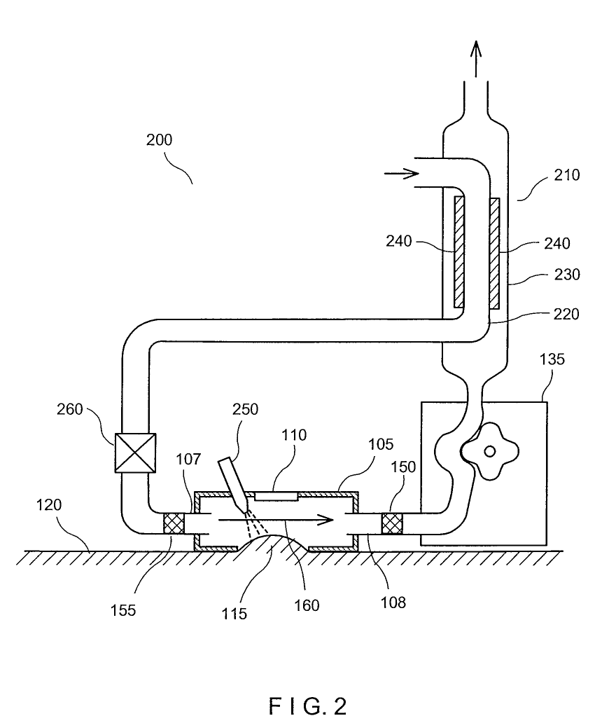

A further exemplary embodiment of a cooling apparatus 200 in accordance with the present disclosure in shown in FIG. 2. Similar to the exemplary cooling apparatus 100 shown in FIG. 1, the apparatus 200 can include the housing 105 with the inlet duct 107 and the outlet duct 108 attached thereto, the window 110, the low-pressure source 135, the inlet filter 155, and the outlet filter 150. The exemplary cooling apparatus 200 can also include a heat exchange arrangement 210, which can include an inner duct 220, an outer duct 230, and a chilling arrangement 240. The exemplary cooling apparatus 200 can also be provided with a spray nozzle 250 attached to the housing 105, and an optional inlet valve 260 provided in communication with the inlet duct 107.

The operation of the exemplary cooling apparatus 200 is similar to that described above for the exemplary cooling apparatus 100 shown in FIG. 1. The low-pressure source 135 may be configured to pull gas through the outlet duct 108 from the volume enclosed by the housing 115. Further gas can be pulled into this volume through the inlet duct 107, creating a gas flow 160 that cools the target region 115.

In this exemplary embodiment, the inlet duct 107 can be connected to the inner duct 220 of the heat exchange arrangement 210. The inner duct 220 can have the form of, for example, one or more tubes or conduits having any of a variety of cross-sectional shapes, e.g., round, rectangular, oval, or the like. The chilling arrangement 240 can be provided in contact with the inner duct 220 to cool gas passing therethrough. The chilling arrangement 240 can include, for example, one or more Peltier elements having the cooling side thereof in contact with and/or forming a portion of the wall of the inner duct 220, or any other suitable heat exchange device configured to cool the inner duct 220. Gas which is cooled by the chilling arrangement 240 can be pulled through the inlet duct 107 by the low-pressure source 135 to create the flow 160 of the cooled gas that flows over the target region 115.

In certain exemplary embodiments, an outer duct 230 can be provided in the heat exchange arrangement 210 that is connected to the outlet duct 108 and the low-pressure source 135. For example, the low-pressure source 135 can be situated between the outlet duct 108 and the outer duct 230 as shown in FIG. 2. Other configurations may be used, e.g., the outer duct 230 may be provided between the outlet duct 108 and the low-pressure source 135. The outer duct 230 can surround at least a portion of the chilling arrangement 240 and/or a portion of the inner duct 220 as shown in FIG. 2. For example, the outer duct 230 can have the form of a tube or other passageway that surrounds both the inner duct 220 and the chilling element 240.

Gas pulled through the outlet duct 108 by low-pressure source 135 can flow through the outer duct 230 and over an outer surface or a portion of the chilling arrangement 240, which can enhance the cooling efficiency of the chilling arrangement 240. For example, if the chilling arrangement includes a Peltier element, gas flowing over the hot side of the Peltier element through outer duct 230 can facilitate a removal of the heat extracted from the inner duct 220 by the cold side of the Peltier element. The gas flowing through the outer duct 230 may still be slightly cooled after flowing through the housing 105, which can further increase the cooling efficiency of the heat exchange arrangement 210.

The exemplary cooling apparatus 200 can also include the spray nozzle 250 coupled to the housing 105. The spray nozzle 250 can be configured to controllably direct a spray of liquid towards the target area 115. The gas flow 160 can increase the evaporation rate of the liquid on the surface of the target area 115, and thereby provide enhanced surface cooling by evaporation. The spray of liquid may be continuous, periodic, or pulsed. For example, the spray nozzle 250 can be vacuum-activated, such that a spray of liquid is produced while the low-pressure arrangement 135 produces a flow 160 of gas within the volume enclosed by the housing 105.

The liquid provided by or through the spray nozzle 250 can be water, alcohol, or any other liquid or combination of liquids that will evaporate when exposed to the gas flow 160 to provide enhanced cooling. This liquid can also include other substances which may provide a beneficial effect to the target area before, during, and/or after treatment by exposure to electromagnetic radiation. Such substances can include analgesics (e.g., a lidocaine solution), antibiotics, or other biologically active agents.

The exemplary cooling apparatus 200 can also include the inlet valve 260 provided in the inlet duct 107. The inlet valve 260 can be configured to controllably start, stop, and/or regulate the flow of gas into the volume contained below the housing 105. For example, the inlet valve 260 can be an electronically-actuated gate valve, a rotating wheel having cut-outs that alternately open and block the flow of gas through the inlet duct 107, or other valve mechanisms. A rotating wheel which includes regions having different densities of openings or passages therethrough can also be provided, which can generate a smooth variation of pressure changes and flow beneath the housing 105 as the wheel rotates and partially obstructs the inlet duct 107.

Control circuitry for the inlet valve 260 can operate the inlet valve 260 so as to obtain different pulse frequencies and flow patterns in order to achieve a desired flow response (such as, e.g., to match a resonant frequency of the tissue surface in the target region 115). Further, circuitry may be provided to adjust other parameters associated with the apparatus in order to achieve desired flow characteristics. Such exemplary parameters can include, for example, the valve diameter, the distance of flow over the target region 115 (e.g., based on the length of the housing 105 and/or the size of an opening provided on a lower surface of the housing 105), etc. For example, vibration of the tissue surface induced by flow can provide further analgesia in accordance with gate control theory, where additional sensation associated with the vibration “occupies” local nerve endings and reduces their ability to detect and transmit pain signals.

An optional vibration detector can also be provided to detect vibration of the skin surface in the target region 115. The vibration detector can include, e.g., a lateral diode laser or other light source together with one or more photodiode detectors which are configured to measure the vibration elevation and frequency of the skin surface. The exemplary vibration detector can be coupled to a controller in a feedback configuration which can control the operation of the inlet valve 260, the low-pressure source 135, and/or other components of the exemplary cooling apparatus 200 to achieve and/or maintain a desired vibration of the tissue in the target region 115.

The inlet valve 260 can be controlled to allow or facilitate the pulses of gas to flow over the target region 115, e.g., in-between pulses of electromagnetic energy which may be applied to the target region 115. The inlet valve 260 can also be provided in communication with the spray nozzle 250, such that a brief spray of liquid onto the target area 115 is followed by a pulse of gas flowing over the target region 115 to provide an intermittent evaporative cooling. The timing and duration of the liquid spray and gas pulses can be selected to provide a desired cooling of the target region 115.

The inlet valve 260 can also be operated in a continuous pulsed mode such that it allows or facilitate a continuous stream of gas pulses to be pulled through the inlet duct 107 by the low-pressure source 135 and flow through the housing 115. This can be achieved, for example, by rapidly cycling the inlet valve 260 between open and closed states, while the low-pressure source 135 is operating. The duration of the open and closed states, and the frequency of switching between the states, can be selected to achieve a desired pulsed flow of gas over the target region 115. For example, the pulse duration can be selected as the length of the cooled portion of the inner duct 220 divided by the flow velocity when the valve is opened. This exemplary procedure can provide a series of pulses, where the gas in each pulse is obtained substantially from the cooled portion of the inner duct 220. Other criteria may be used to determine the valve operation parameters based on resultant cooling and flow behaviors for particular treatments.

The gas entering the inlet duct 107 from the cooling enclosure 140 or the inner duct 220 of the heat exchange arrangement 210 can be, for example, air that is pulled in from the environment surrounding the exemplary cooling apparati 100, 200. Alternatively, such gas can be provided from a controlled source, such as a gas canister. A controlled gas source can allow or facilitate treatment of the target region 115 to be performed under a specified environment. For example, a low-oxygen or oxygen-free gas mixture, a gas containing predetermined amounts of beneficial substances, or any other desired gas composition can be provided.

A further exemplary embodiment of a cooling apparatus 400 in accordance with the present disclosure in shown in FIG. 4. Similar to the exemplary cooling apparati 100, 200 shown in FIGS. 1 and 2, respectively, the exemplary apparatus 400 can include the housing 105 with the inlet duct 107 and the outlet duct 108 attached thereto, and the window 110.

A distal portion of the energy source 130, e.g., an end of a waveguide or a casing enclosing such waveguide, the aperture of the laser or the IPL, a portion of an energy delivery handpiece or the like, can be mechanically coupled to the housing 105 of the exemplary cooling apparatus 400. This coupling can be rigid, or it can allow or facilitate an angular movement of the energy source 130 relative to the housing to enable the electromagnetic energy 125 to be directed towards various portions of the target region 115. For example, if the distal portion of the energy source 130 is rigidly coupled to the housing 105, the electromagnetic energy 125 can be directed towards various portions of the target region 115 by translating the entire housing 105 relative to the target region 115. Alternatively, conventional optical components or the like associated with the energy source 130 can be used to alter the direction of the electromagnetic energy 125 being emitted from the distal portion of the energy source 130.

The low-pressure source 410 that may be used with the exemplary cooling apparatus 400 (or with other exemplary embodiments of the present disclosure) can be, for example, a container or reservoir enclosing a gas under vacuum or low pressure, e.g., an evacuated container or the like. The low-pressure source 410 may be provided in communication with the outlet duct 108. A valve 420 can be provided between the outlet duct 108 and the low-pressure source 410. Withdrawal of gas through the outlet duct 108 from the volume enclosed by the housing 105, and thus flow of the gas over the target region 115 of the tissue 120, can be controlled or regulated, e.g., by operation of the valve 420. For example, debris or effluent that may be produced within the enclosed volume of the housing 105 can be withdrawn through the outlet duct 108 and into the low-pressure source 410. After use, the low-pressure source 410 may optionally be discarded or cleaned and re-used.

In a further aspect, exemplary embodiments of the present disclosure can provide a method for cooling a surface which includes providing a housing to cover a portion of the surface and at least partially enclose a volume between the housing and a surface region to be cooled. A gas can be drawn through the enclosed volume to generate a flow of the gas over the surface region. This flow may provide convective cooling of the surface region.

The gas can be withdrawn from the enclosed volume through one or more outlet ducts connected to the housing, and further gas can enter the enclosed volume through one or more inlet ducts connected to the housing. The gas can be air, air mixed with one or more additional components (such as a further gas or a vaporized substance), and/or it can be any other gas which may be provided through the inlet ducts.

In further embodiments of the exemplary cooling method, at least a portion of the gas provided to the inlet duct can be cooled before it enters the enclosed volume. Such cooling of the gas can increase the degree and/or efficiency of cooling of the surface.

In still further exemplary embodiments of the cooling method, the surface being cooled can be sprayed with a liquid. Evaporation of the liquid from the surface, which may be enhanced by the flow of gas, can provide additional cooling. A variety of liquids can be used including, e.g., water, alcohol, another liquid which can be evaporated using the gas flow, or a mixture thereof. The liquid spray can be continuous, intermittent, or of a finite duration.

The foregoing merely illustrates the principles of the invention. Various modifications and combinations of the described embodiments and/or elements thereof will be apparent to those skilled in the art in view of the teachings herein. It will thus be appreciated that those skilled in the art will be able to devise numerous techniques which, although not explicitly described herein, embody the principles of the invention and are thus within the spirit and scope of the invention.

What is claimed is:

1. An apparatus for cooling a surface region, the apparatus comprising:
a housing comprising an opening on a lower portion thereof, wherein the housing is configured to at least partially enclose a volume above the surface region;
at least one inlet arrangement at least one of provided on and/or coupled to a first portion of the housing; and
at least one outlet arrangement at least one of provided on and/or coupled to a second portion of the housing,
wherein the inlet and outlet arrangements are provided to facilitate a flow of a gas over the surface region when a low-pressure source is connected to the at least one outlet arrangement.

2. The apparatus of claim 1, wherein at least third portion of the housing is configured to facilitate an electromagnetic radiation to pass therethrough without damaging the housing.

3. The apparatus of any of claims 1 and/or 2, wherein the electromagnetic radiation is provided by at least one of a laser, a flashlamp, or an intense pulsed light source.

4. The apparatus of any of claims 1-3, wherein the housing further comprises at least one window configured to facilitate a visual observation of the surface region.

5. The apparatus of any of claims 1-4, wherein the housing comprises a transparent material configured to facilitate a visual observation of the surface region.

6. The apparatus of any of claims 1-5, further comprising at least one first filter arrangement provided in communication with the at least one outlet arrangement.

7. The apparatus of any of claims 1-6, further comprising at least one second filter arrangement provided in communication with the at least one inlet arrangement.

8. The apparatus of any of claims 1-7, further comprising an inlet valve arrangement provided in communication with the at least one inlet arrangement, and configured to control the flow of gas entering the enclosed volume.

9. The apparatus of any of claims 1-8, further comprising an outlet valve arrangement provided in communication with the outlet duct that is configured to control the flow of gas exiting the enclosed volume.

10. The apparatus of any of claims 8 and/or 9, wherein at least one of the inlet valve arrangement and/or the outlet valve arrangement is configured to generate a pulsed flow of the gas through the enclosed volume.

11. The apparatus of any of claims 1-10, further comprising a cooling arrangement provided in communication with the at least one inlet arrangement, wherein the cooling arrangement is configured to cool the gas before the gas enters the enclosed volume through the at least one inlet arrangement.

12. The apparatus of claim 11, wherein the cooling arrangement comprises at least one of a Peltier device, a phase-change medium, and/or a cooling duct configured to facilitate a cooled fluid to flow therethrough.

13. The apparatus of claim 12, wherein the phase-change medium comprises at least one of ice and/or dry ice.

14. The apparatus of any of claims 11-13, wherein the cooling arrangement is configured to provide at least a portion of the gas exiting the enclosed volume through the at least one outlet arrangement in a thermal communication with at least a portion of the gas entering the at least one inlet arrangement.

15. The apparatus of any of claims 1-14, further comprising a spray arrangement coupled to the housing, wherein the spray arrangement is configured to spray a fluid onto at least one portion of the surface region.

16. The apparatus of claim 15, wherein the spray arrangement comprises at least one spray nozzle.

17. The apparatus of any of claims 15 and/or 16, wherein the fluid comprises at least one of alcohol and/or water.

18. The apparatus of any of claims 15-17, wherein the spray arrangement is configured to be activated by the flow of the gas within the enclosed volume.

19. The apparatus of any of claims 1-18, wherein the apparatus further comprises the low-pressure source.

20. The apparatus of claim 19, wherein the low-pressure source comprises at least one of a vacuum pump, a positive displacement pump and/or an evacuated container.

21. The apparatus of any of claims 1-20, wherein the housing further comprises a hole, and wherein the hole is configured to facilitate a delivery portion of an energy source to be connected to the housing such that energy delivered by the energy source impinges on the surface region without passing through a portion of the housing.

22. The apparatus of claim 21, wherein the delivery portion comprises at least one of a waveguide, an optical fiber and/or a handpiece.

23. The apparatus of any of claims 21 and/or 22, wherein the hole is configured to facilitate the delivery portion to be movably coupled to the housing.

24. The apparatus of any of claims 1-23, wherein the surface region is part of a biological tissue.

25. The apparatus of any of claims 1-24, wherein the inlet and outlet arrangements include ducts.

26. A method of cooling a biological surface region, the method comprising:
at least partially enclosing a volume above the surface region; and
drawing a gas through the enclosed volume to cool the surface region.
